(19) United States
(12) Patent Application Publication
(10) Pub. No.: US 2023/0093672 A1
(43) Pub. Date: Mar. 23, 2023

(54) PROCESS FOR SYNTHESIS OF DIMETHYL ETHER

(71) Applicants: AMOL CARBONS PRIVATE LIMITED, Pune (IN); INDIAN OIL CORPORATION LIMITED, Mumbai (IN)

(72) Inventors: Anand Janardan APTE, Pune (IN); Rajesh Muralidhar BADHE, Faridabad (IN)

(21) Appl. No.: 17/905,033

(22) PCT Filed: Feb. 26, 2021
(86) PCT No.: PCT/IN2021/050188

§ 371 Date: Aug. 25, 2022

(30) Foreign Application Priority Data
Feb. 27, 2020 (IN) 202021008375

(51) Int. Cl. C07C 41/01

(52) U.S. Cl. CPC C07C 41/01

(57) ABSTRACT

The present invention depicts a method for one pot synthesis of dimethyl ether from syngas in a simple and economical manner. The process (500A, 500B, 600) has advantages of reducing the requirement of refrigeration and at the same time producing a ready to use product. The process (500A, 500B, 600) includes the steps of separating carbon dioxide from a first stream (512, 612) comprising syngas to produce a second stream (522, 622), reacting the second stream (522, 622) in the presence of a catalyst to produce a third stream (532, 632), cooling the third stream (532, 632) to a temperature in a range from 10° C. to 40° C. to produce a fourth stream (542, 642), and washing and conducting a phase separation of the fourth stream (542, 642) to produce a product comprising at least 10% by volume of dimethyl ether.

### FIELD OF THE INVENTION

The present invention relates to production of dimethyl ether from syngas in a simple and economical manner. Specifically, the invention relates to a process for producing a ready to use dimethyl ether product without the requirement of refrigeration in the process.

### BACKGROUND OF THE INVENTION

The dimethyl ether is being increasingly viewed as the fuel of the future on account of its many favorable attributes. It is a clean burning fuel with physicochemical properties comparable to Liquid petroleum gas (LPG) and cetane number higher than high speed diesel. It, therefore, can serve as an effective substitute for LPG as well as diesel. Furthermore, it can be made from a wide variety of feedstocks such as coal, natural gas, agricultural wastes etc. and it can be blended with LPG and diesel in any proportions desired.

Presently, dimethyl ether (DME) is manufactured by a two-step process. First methanol is synthesized from synthesis gas by reaction 1.

CO+2 H₂→CH₃OH ΔH −90.29 kJ/mole (1)

DME is synthesized from methanol by reaction 2.

2 CH₃OH→CH₃OCH₃+H₂O ΔH −23.41 kJ/mole (2)

Potential of DME to replace petroleum is recognized and is manufactured at a large scale from local coal deposits. The two-step methanol to DME conversion is a well-established process. These two reactions viz. methanol synthesis and methanol dehydration reactions are carried out in two separate reactors under different operating conditions. Methanol synthesis is optimally carried out at a temperature of 200-300° C., and at a pressure of 50-80 bar using CuZnOAl₂O₃ catalyst, with long catalyst life of 2 to 5 years. The optimum conditions for methanol to DME conversion are temperature of 100 to 300° C. and pressure of 20 bar, using solid acid catalyst such heteropoly acids (temperature 100-200° C.), HZSM-5 (around 200° C.) or γ-Al₂O₃ (above 250° C.) with long catalyst life.

This two-step process has severe thermodynamic limitations as it suffers from low per pass conversion ca. 20%, poor energy efficiency and requires syngas rich in hydrogen (H₂/CO=2:1). This hydrogen rich syngas is especially difficult to obtain from feedstocks such as coal and agricultural waste. By combining reaction 3 with reactions 1 & 2:

CO+H₂O→CO₂+H₂ ΔH −48.46 kJ/mole (3)

DME can be directly manufactured from syngas deficient in hydrogen (H₂/CO≈1). The low hydrogen syngas can be readily manufactured from coal and agricultural waste. The single step process is desirable due to multiple benefits: High conversion efficiency 60% to 77%, high energy efficiency and freedom to use lean syngas deficient in hydrogen. The overall process can be represented as follows:

3 CO+3 H₂→CH₃—O—CH₃+CO₂ ΔH −254.4 kJ/mole DME (4)

To carry out this DME synthesis in a single step from syngas deficient in hydrogen is the main objective of many researchers. A single pot DME process is carried out by combining methanol synthesis catalyst with a methanol dehydration catalyst. Most commercial methanol synthesis catalysts are also active for shift reaction (reaction 3 above). In short two catalysts are needed for single pot synthesis.

JFE, Japan were the first to test single pot DME process. The process used in the plant is as shown in FIG. 1. FIG. 2 shows typical material balance for the plant. One of the challenges in integrating the methanol synthesis and dehydration is in recovering the highly volatile DME from raw product mixture containing CO, H₂, methanol, DME, byproducts (alkenes & water) and CO₂. The JFE used cold (−40° C.) methanol to remove all components except CO and H₂ from the raw product gas. The liquid raw product is then distilled to remove CO₂. A second distillation recovers DME as a top product and crude methanol containing water as bottom product. A third distillation is required to recover methanol as product. Refrigeration at −40° C. is required for this process. A similar process is described in patent DE 4,222,655. Several variations on this basic theme were tested, all requiring large refrigeration.

EP 0871602 B1 patent describes cooling raw product, separating liquid followed by recycling the gas to the reactor as shown in FIG. 3. The material balance shown in examples illustrate the disadvantage of this scheme, the sub stream coming from the separator and joining the syngas stream in the FIG. 3 contains as much as 36.4% by weight of the final product. Feeding the reactor with such a large quantum of final product leads to wastage.

EP 2028173 A1 describes another method, wherein the raw products from DME synthesis are washed with potassium carbonate solution followed by adsorption on molecular sieve. Following these two treatments all of the methanol as well as DME is still with unreacted syngas and its concentration is only 35%. The raw products are then purified by distillation. Distillation removes water and methanol as bottom products. Recovery of DME from unreacted syn-gas requires refrigeration to condense the volatile DME product. The recycled syngas still contains significant quantity of DME.

EP 2070905 A1 describes a scheme whereby dimethyl ether of polyethylene glycol (Trade name Selexol) is used as scrub liquid. The advantage of this scrub liquid is that it has high solubility of CO₂, DME and methanol and water. FIG. 4 shows the overall processing scheme for this method. Reactor 2 is the main DME synthesis reactor, the raw product contains unreacted CO, H₂, and reaction products DME, Methanol, water, and CO₂ supplied by the stream 1. The raw product is cooled at cooler 5 thereby condensing a majority of methanol and water, removing the condensate as stream 6. Selexol wash at 8 removes a majority of DME and CO₂ and is carried off with Selexol in stream 10. Stream 10 is flashed in stages to remove CO₂ and DME. Lean Selexol is recycled to scrubber as stream 16. The challenge with this scheme is that the equipment in box 14 are more complex than just flash vessels as condensation of DME requires refrigeration. Another disadvantage is the solubility of CO₂ in DME, especially at cold temperatures and/or high pressure. Most of the above-described processes require refrigeration as well as multiple steps to recover the product DME.

### OBJECTIVES OF THE INVENTION

One of the objectives of the present invention is to provide an economical process requiring minimal cooling and generating a product in minimum steps.

Another objective of the present invention is to produce a final product in a single step of separating the multiple products obtained during DME synthesis.

Yet another objective of the present invention is to provide a two-step method to generate pure DME.

One more objective of the invention is to produce a dispensing ready product for the transportation sector.

### SUMMARY OF THE INVENTION

Aspects of the present disclosure depict a method for one pot synthesis of dimethyl ether from syngas in a simple and economical manner The process has advantages of eliminating the requirement of refrigeration and at the same time producing a ready to use product. The process includes the steps of separating carbon dioxide from a first stream comprising syngas to produce a second stream, reacting the second stream in the presence of a catalyst to produce a third stream, cooling the third stream to a temperature in a range from 10° C. to 40° C. to produce a fourth stream, and washing and conducting a phase separation of the fourth stream to produce a product comprising at least 10% by volume of dimethyl ether. The second stream produced is lean in carbon dioxide content. The third stream includes dimethyl ether, methanol, water, carbon monoxide, carbon dioxide, and hydrogen.

### BRIEF DESCRIPTION OF THE FIGURES

These and other features, aspects, and advantages of the exemplary embodiments can be better understood when the following detailed description is read with reference to the accompanying drawings in which like characters represent like parts throughout the drawings, wherein:

FIG. 1 illustrates a schematic process used in the plant of a prior art;

FIG. 2 shows a typical material balance for the plant used in FIG. 1;

FIG. 3 illustrates a process for cooling raw product, separating liquid and recycling the gas to the reactor as illustrated in EP 0871602 B1;

FIG. 4 shows a process whereby dimethyl ether of polyethylene glycol is used as scrub liquid, as illustrated in EP2 070 905 A1;

FIG. 5A illustrates a process for the production of dimethyl ether from syn-gas, in accordance with an embodiment of the present invention,

FIG. 5B illustrates a process for the production of dimethyl ether from syn-gas with a slight variation from 5B, in accordance with an embodiment of the present invention; and

FIG. 6 illustrates another process for the production of dimethyl ether from syn-gas, in accordance with an embodiment of the present invention.

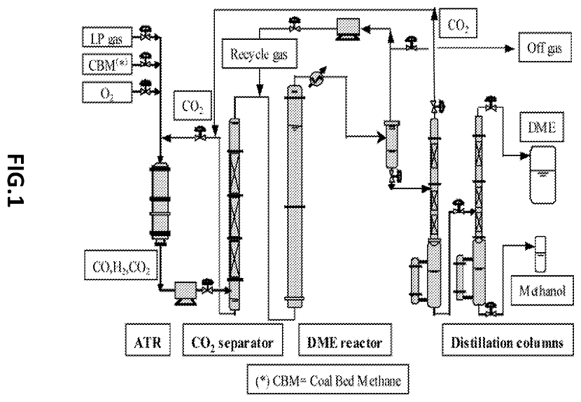

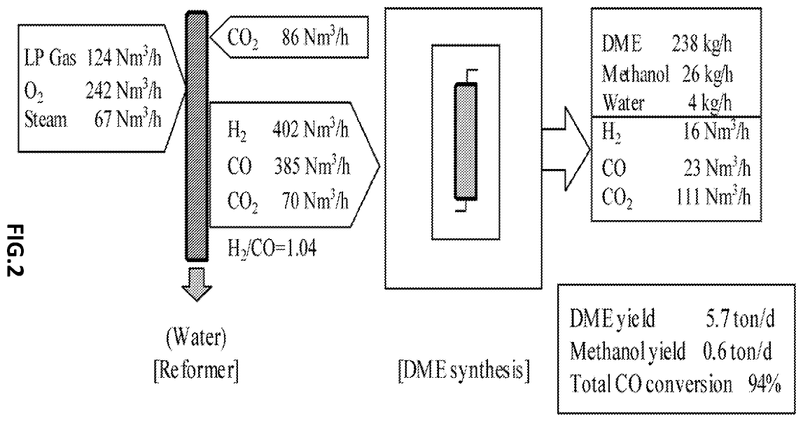

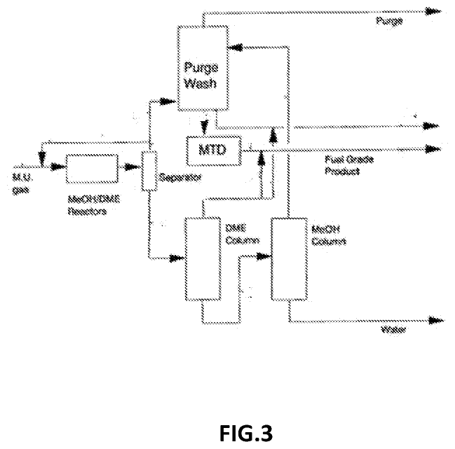

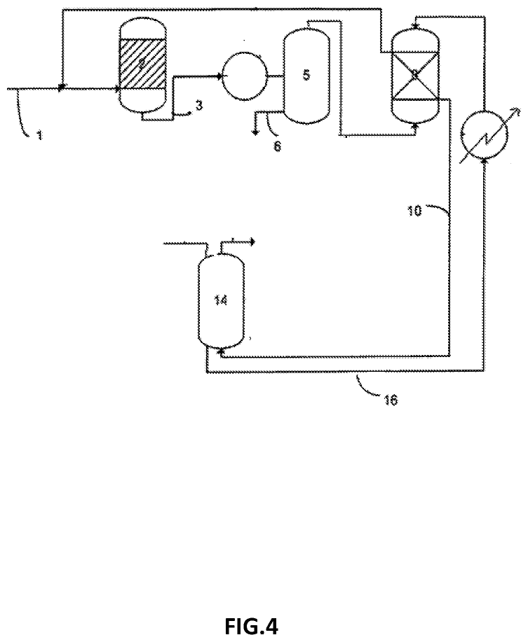

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention.

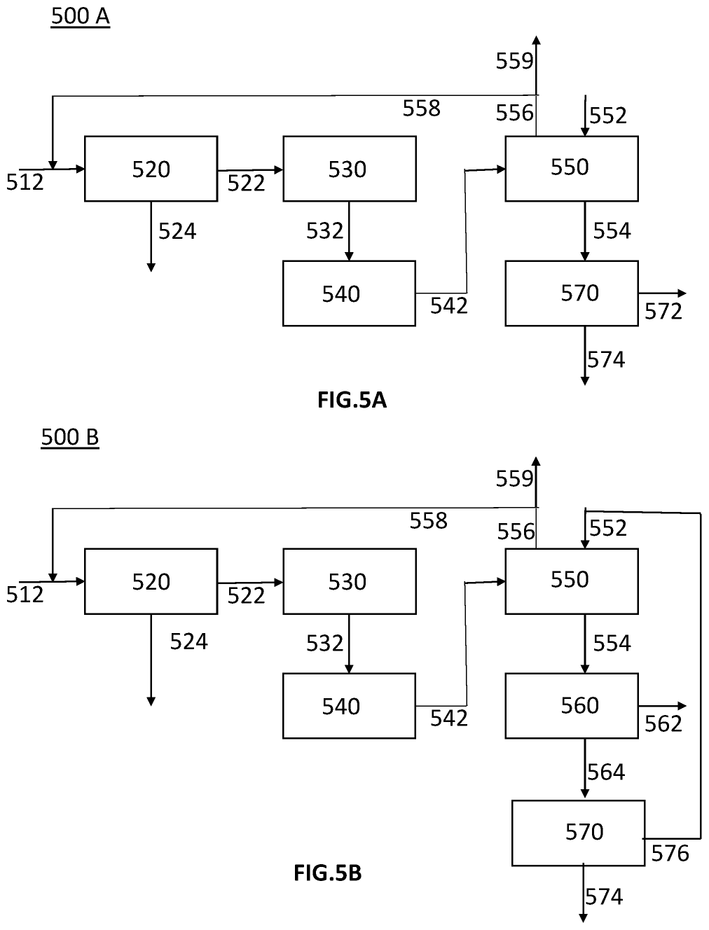

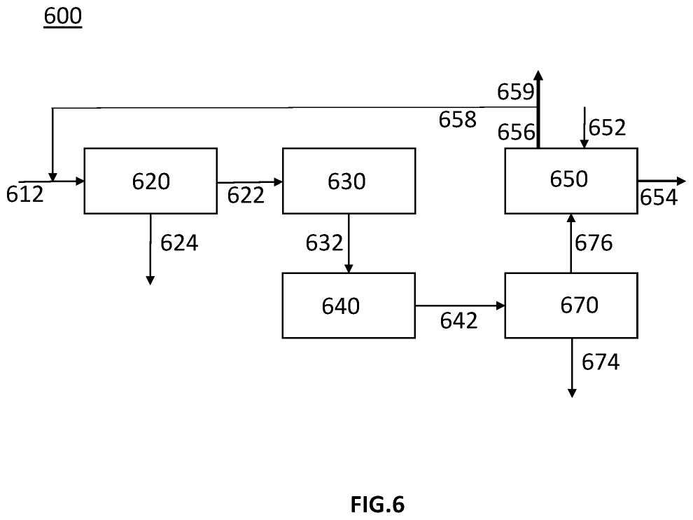

Aspects of the present disclosure includes a method for one pot synthesis of dimethyl ether from syngas as shown in FIGS. 5A, 5B, and 6. FIGS. 5A and 5B represent one pot synthesis process 500A and 500B respectively, for the synthesis of dimethyl ether. In FIGS. 5A and 5B, the stream 512 represents syngas from a suitable source such as biomass gasifier, auto thermal reformer of biogas or natural gas, coal gasifier etc. The syngas includes CO, hydrogen, and CO₂. The syngas contains CO and hydrogen in a specific proportion. The proportion of CO and hydrogen may vary depending on the selected process of producing syngas.

The first step of DME synthesis by this method includes separating carbon dioxide from the first stream 512. In the first step, the syngas is fed in to a carbon dioxide stripping system 520. In some embodiments, the syngas stream 512 is mixed with a recycled gas stream before feeding to the carbon dioxide stripping system 520. In some embodiments, the recycled gas stream 558 may be part of a stream 556 obtained from a scrubber that is used to wash the reaction products of the dimethyl ether synthesis. The carbon dioxide stripping system 520 may use any convenient method such as amine wash, hot pot or benfield, selexol or other acid gas removal system. In some embodiments, the first stream 512 is subjected to a water wash prior to the carbon dioxide stripping.

Notably, the carbon dioxide stripping system 520 removes CO₂ prior to feeding the syngas, or the syngas along with the recycled stream, into the DME synthesis reactor 530. Thus, the second stream 522 is essentially lean in CO₂ content. In some embodiments, the CO₂ content in the second stream 522 is less than 2 vol. % of the second stream 522. In some embodiments, the content of CO₂ in the second stream 522 is less than 1 vol. % of the second stream 522. The CO₂ stripping is especially advantages as it increases the partial pressure of reactants CO and H₂ in the second stream 522, and thereby specifically aids effective reaction between the reactants of the second stream 522 in the reactor 530 leading to the efficient production of DME. The stripped CO₂ may be removed as a CO₂ stream 524.

The reactants in the second stream 522 react at the reactor 530 in the presence of one or more catalysts. The reactor 530 may have any advantageous design to aid increased reactions of the syngas and DME production. The reaction in the reactor 530 may be conducted at an elevated temperature and/or pressure. In some embodiments, the reaction in the reactor 530 includes reacting the second stream 522 at an enhanced temperature and at a pressure higher than the atmospheric pressure. In some embodiments, the temperature of the reactor 530 during the reaction is in a range from 200° C. to 300° C. In some embodiments, the pressure exerted at the time of reaction is in a range from 20 bars to 30 bars. In some embodiments, two or more catalysts are used for the production of DME in the reactor 530.

The third stream 532 emerging from the reactor 530 may include unreacted CO, H₂, and reaction products DME, methanol, water, and CO₂. These products are cooled in one or more stages in one or more coolers 540 to a temperature near room temperature as achievable using cooling water to produce a cooled fourth stream 542.

It is to be noted that the third stream 532 is cooled at cooler 540 by just using a cooling water. Any other cooling stream may be used for the cooling purpose, but there is no need of cooling by a chilled or refrigerated coolant to cool the contents of the third stream. In all embodiments, the step of cooling does not include chilling or refrigerating. In some embodiments, the third stream is cooled to a temperature greater than 20° C. to produce a fourth stream. Thus, effectively, the temperature of the cooled stream 542 is not less than 10° C. Further, the temperature of the cooled stream is no more than 40° C. as determined by available temperature of cooling water/air cooling which depends on the location and season at any given point of time. Not using a chiller or refrigerator for the cooling step is a significant energy saver in the disclosed method of DME synthesis and in contrast to the hereto known methods of one pot DME synthesis. In some embodiments, the temperature of the cooled stream 542 is in a range from 15° C. to 35° C.

The cooled stream 542 emerging from the cooler 540 is further subjected to washing and product separation to produce a product comprising at least 10% by volume of dimethyl ether. In some embodiments, the step of washing includes using a hydrocarbon solvent scrubber. The product separation may include condensation, phase separation, distillation, or any combinations of any of these methods. The cooled stream may be subjected to washing first and then phase separation or vice versa. FIGS. 5A and 5B represent process steps where washing of the cooled stream 542 is conducted prior to the phase separation. FIG. 6 represents process steps where the phase separation is conducted prior to washing the cooled stream 642, while all the prior steps up to cooling the products may be same as different from that depicted in FIGS. 5A and 5B.

The final product stream 572 obtained after the washing and phase separation steps includes at least 10 wt. % of dimethyl ether. In some embodiments, the product includes dimethyl ether in a quantity greater than 20 wt. % of the product stream. In some specific embodiments, the quantity of dimethyl ether in the final product stream exceeds 30 vol. % of the product stream.

In FIG. 5A and 5B, the cooled stream 542 is scrubbed in a scrubber 550 to wash down the DME. The scrubbing process in the scrubber 550 may use a solvent supplied though a scrubber solvent inlet 552. In some embodiments, hydrocarbon solvent is used as scrub liquid. The scrubbed stream 554 is subjected to phase separation in a phase separator 570.

In some embodiments, the phase separator 570 is a liquid phase separator. In some other embodiments, the phase separator 570 may be a liquid-gas phase separator. The phase separator 570 separates the final product stream 572 from the other liquids present in the scrubbed stream 554. Other liquids in the scrubbed stream may form a byproduct stream 574 and may include water, methanol, and some amount of DME. The scrubber 550 may generate a scrubber byproduct stream 556 as a sixth stream that may include CO, hydrogen, and CO₂. This scrubber byproduct stream 556 may be recycled to combine with the first stream containing syngas to increase yield of DME product. A purge stream 559 may be taken out of stream 556, to avoid build-up of undesirable component in recycle.

The process 500B in FIG. 5B includes at least one more step than the process 500A depicted in FIG. 5A. In the process 500B, the scrubbed products are subjected to distillation in a distillation unit 560 to produce a product stream 562. A pure DME may be obtained as the product stream 562. The remaining substances of the distillation forms a byproduct stream 564 and may contain the solvents used in the scrubber, water, and methanol. This byproduct stream 564 may be subjected to phase separation in the phase separator 570 to further separate the solvents used in the scrubber from the water and methanol. Further, another byproduct stream 576 may be cycled to the scrubber 550 to be used for washing the stream 542.

FIG. 6 depicts a variation of the process shown in FIGS. 5A and 5B. In the process 600 depicted in FIG. 6, the first stream 612 is stripped off the CO₂ in the CO₂ separator 620 to remove a CO₂ stream 624, and the CO₂ stripped second stream 622 is reacted at the reactor 630 to produce the reaction products. The third stream 632 containing the reaction products and unreacted solvents are cooled in the cooler 640. The cooled stream 642 is directed to a phase separator 670 before washing. In some embodiments, the phase separator 670 may be a gas—liquid separator. The gas part of the separated products in the phase separator 670 is directed as a gas stream 676 to the scrubber 650. In some embodiments, the stream 676 is essentially free of water and methanol. The stream 676 is washed by a suitable hydrocarbon solvent. If diesel used in transportation sector is the target product, then the hydrocarbon solvent can be HSD, high speed diesel. Stream 654 in this case represents the final product. If the hydrocarbon solvent used for scrubbing is HSD, a blended DME-diesel fuel is obtained as the final product 654. The scrubbed gas at stream 656 now contains only CO, H₂ and CO₂ and it can be recycled as recycled stream 658 to be combined with the stream 612. A part of stream 656 may be vented to as a vent stream 659 prevent build-up of undesirable components. In some embodiments, at least a part of the sixth stream 656 is recycled as the recycled stream 658 to combine with the first stream 612 for enhancing conversion of the syngas to dimethyl ether. In a specific embodiment, more than 60% of the sixth stream 656 is recycled to combine with the first stream 612. The condensed methanol and water separated at the phase separator 670 are removed as stream 674. In the scrubber 650, the dimethyl ether is washed with hydrocarbon solvent and produced as a final product stream 654.

### EXAMPLE

In an example embodiment, the above-described method generating DME-diesel mixture as main product was experimented over a 21-hour trial run. The plant used for the trial run was a comparatively smaller unit than a standard unit and had a small capacity. Further, the CO₂ separation was not conducted in the trial run. As the size was comparatively smaller, the raw product stream reached near ambient temperature without the need for active cooling. Diesel at the rate of 15 ml/minute was fed to the scrubber and the stream containing methanol and water was collected in a tank. The tank contained the total liquid product produced. The break-up of the liquid product was as follows:
1. Wash diesel fed to the tower: 12.1 kg
2. Total DME product: 7.9 kg
3. Total aqueous product: 1.4 kg
4. DME concentration in aqueous product: 30%
5. DME concentration in Diesel: 39%
6. Fraction of total product collected by Diesel: 92.4%

This example clearly shows the advantage of hydrocarbon wash as a product purification step. 92.4% of total product was recovered by hydrocarbon wash. Only less than 8% product needs a second stage of distillation to fully recover product DME.

There is no requirement for any engine modification for the DME dissolved in HSD up to 20% by weight. Only the storage tank and fuel filter needs modification for accepting a high vapor pressure fuel. Thus, any DME in diesel up to 20% is a marketable product. The petroleum company may dispense the DME diesel mixture in an LPG type dispenser. If DME content is kept as low as 1-3%, then even the tank and fuel filter need not be changed, and no change is required at the fuel dispensing end. If pure DME is the target product, then the solvent can be a light oil such as Naphtha. Pure DME can be easily distilled out, as relative volatility of DME to Naphtha is more than 100.

The disclosed process for the synthesis of DME provides an economical process that requires minimal cooling and generates the DME product in minimum steps. The disclosed process provides a two-step method to generate pure DME, and separates the multiple products obtained during DME synthesis using a single separation step. Further, the disclosed process produces a dispensing ready product for the transportation sector.

While the invention is susceptible to various modifications and alternative forms, specific embodiment thereof are described in details above. It should be understood, however that it is not intended to limit the invention to the particular forms disclosed, but on the contrary, the invention is to cover all modifications, equivalents, and alternative falling within the scope of the invention as defined by the appended claims.

What is claimed is:

1. A process for one pot synthesis of dimethyl ether, the process comprising:
separating carbon dioxide from a first stream comprising syngas to produce a second stream, wherein the second stream is lean in carbon dioxide content;
reacting the second stream in the presence of a catalyst to produce a third stream comprising dimethyl ether, methanol, water, carbon monoxide, carbon dioxide, and hydrogen;
cooling the third stream to temperature in a range from 10° C. to 40° C. to produce a fourth stream;
washing and conducting a phase separation of the fourth stream to produce a product stream comprising at least 10% by volume of dimethyl ether.

2. The process according to claim 1, comprising separating the carbon dioxide as a carbon dioxide stream from the first stream through a water wash followed by carbon dioxide stripping.

3. The process according to claim 1, comprising reacting the second stream at an enhanced temperature in a range from 200° C. to 300° C. and pressure in a range from 20 bar to 30 bar in the presence of two or more catalysts.

4. The process according to claim 1, wherein the step of cooling does not comprise chilling or refrigerating.

5. The process according to claim 1, wherein the step of washing comprises using a hydrocarbon solvent scrubber producing a sixth stream comprising carbon monoxide, carbon dioxide, and hydrogen.

6. The process according to claim 5, comprising recycling at least a part of the sixth stream as a recycling stream to combine with the first stream for enhancing conversion of the syngas to dimethyl ether.

7. The process according to claim 6, wherein more than 60% of the sixth stream is recycled to combine with the first stream.

8. The process according to claim 1, comprising removing methanol and water from a fifth stream through condensation, phase separation, distillation, or combinations thereof

9. The process according to claim 1, wherein the content of carbon dioxide in the second stream is less than 1% of the total contents of the second stream.

10. The process according to claim 1, comprising washing the fourth stream before conducting the phase separation.
